(19) United States
(12) Patent Application Publication
(10) Pub. No.: US 2021/0123899 A1
(43) Pub. Date: Apr. 29, 2021

(54) METHOD FOR DETERMINING SCALE INHIBITOR CONCENTRATION IN SALT WATER WITH A CALCIUM/MAGNESIUM IONSELECTIVE ELECTRODE

(71) Applicant: SOLENIS TECHNOLOGIES CAYMAN, L.P., George Town (KY)

(72) Inventors: Winfried SCHELLBACH, Ronneburg (DE); Stephan NIED, Ludwigshafen (DE)

(73) Assignee: SOLENIS TECHNOLOGIES CAYMAN, L.P., George Town (KY)

(21) Appl. No.: 16/635,640

(22) PCT Filed: Jul. 26, 2018
(86) PCT No.: PCT/EP2018/070273

§ 371 Date: Jan. 31, 2020

(30) Foreign Application Priority Data
Aug. 4, 2017 (EP) 17184861.7

(51) Int. Cl. G01N 33/18; C02F 5/10; G01N 27/333; C02F 1/44

(52) U.S. Cl. CPC G01N 33/1853; C02F 5/10; C02F 2103/08; C02F 1/44; C02F 2303/22; G01N 27/333

(57) ABSTRACT

The present invention relates to a method for determining a concentration of a scale inhibitor in a salt water comprising an analysis with a calcium/magnesium ionselective electrode of a dialyzed first sample of the salt water, and a dialyzed second sample of the salt water which was supplemented with a known concentration of the scale inhibitor. The invention further relates to a method for inhibiting incrustation in a plant which contains a salt water comprising the steps of adding a scale inhibitor to the salt water at a desired concentration, determining the actual concentration of the scale inhibitor in the salt water as above, and adding further scale inhibitor to the salt water to adjust the desired concentration. The invention further relates to a device for determining a concentration of a scale inhibitor in a salt water by the method above comprising a calcium/magnesium ionselective electrode, a dialyzing unit, and a dosage unit for supplementing the scale inhibitor to the second sample of the salt water.

The present invention relates to a method for determining a concentration of a scale inhibitor in a salt water comprising an analysis with a calcium/magnesium ionselective electrode of a dialyzed first sample of the salt water, and a dialyzed second sample of the salt water which was supplemented with a known concentration of the scale inhibitor. The invention further relates to a method for inhibiting incrustation in a plant which contains a salt water comprising the steps of adding a scale inhibitor to the salt water at a desired concentration, determining the actual concentration of the scale inhibitor in the salt water as above, and adding further scale inhibitor to the salt water to adjust the desired concentration. The invention further relates to a device for determining a concentration of a scale inhibitor in a salt water by the method above comprising a calcium/magnesium ionselective electrode, a dialyzing unit, and a dosage unit for supplementing the scale inhibitor to the second sample of the salt water.

In boilers, piping and other components of water treatment plants, of desalination plants and of water circuits, especially of cooling7 water circuits of industrial plants and of power plants, often scale (incrustation) forms due to the deposition of for example calcium carbonate (CaCO₃, calcite) and magnesium carbonate (MgCO₃). This leads to high costs as frequent cleaning of the boilers, piping and the other components is necessary. Furthermore, the scale can lead to a shortening of the lifetime of the plants as it can lead to severe damages of the boilers, piping and other components of the plants.

To inhibit the scale (incrustation) growth, usually scale inhibitors are added to the water comprised in the water circuits, in the water treatment plants and in the desalination plants. It is assumed, that the scale inhibitors inhibit the formation of scale by colloidal stabilization of precursors, that otherwise would form scale like for example calcite deposit. Scale inhibitors are for example polyacrylic acids and polyaspartic acid. During the inhibition process, the incrustation inhibitor is consumed and therefore its concentration drops. When its concentration is below a certain level the incrustation inhibitor cannot inhibit the growth of scale any longer. Therefore, it is necessary to keep the level of the concentration of the scale inhibitor at a certain value.

To monitor the concentration of the incrustation inhibitor several methods are described in the state of the art. Object was to further improve these methods.

The object was solved by a method for determining a concentration of a scale inhibitor in a salt water comprising an analysis with a calcium/magnesium ionselective electrode of
a) a dialyzed first sample of the salt water, and
b) a dialyzed second sample of the salt water which was supplemented with a known concentration of the scale inhibitor.

The object was also solved by a method for inhibiting incrustation in a plant which contains a salt water comprising the steps of
x) adding a scale inhibitor to the salt water at a desired concentration,
y) determining the actual concentration of the scale inhibitor in the salt water by the method according to the invention, and
z) adding further scale inhibitor to the salt water to adjust the desired concentration.

The object was also solved by a device for determining a concentration of a scale inhibitor in a salt water by the method according to the invention comprising a calcium/magnesium ionselective electrode, a dialyzing unit, and a dosage unit for supplementing the scale inhibitor to the second sample of the salt water.

The scale inhibitor is typically a compound which is suitable for inhibiting the growth of scale in industrial plants. Various scale inhibitors are commercially available.

The scale inhibitor is preferably a polycarboxylic acid (e.g. a polyacrylic acid or a polymaleic acid), or a phophonate. More preferably the scale inhibitor is a polycarboxylic acid, in particular a polyacrylic acid.

The scale inhibitor has usually a number average molecular weight Mₙ of at least 200 g/mol, preferably at least 300 g/mol, and in particular at least 400 g/mol. The scale inhibitor has usually a number average molecular weight Mₙ in the range from 200 to 250000 g/mol, preferably in the range from 800 g/mol to 70000 g/mol, and in particular in the range from 1000 g/mol to 8000 g/mol. The Mₙ may be measured by size exclusion chromatography (SEC) in an aqueous medium using a sodium polyacrylic acid standard and a polyacrylic acid standard for the calibration.

Suitable polycarboxylic acids are polyacrylic acids or polymaleic acids.

Suitable polyacrylic acid (PAA) comprises photopolymers prepared from a monoethylenically unsaturated monocarboxylic acid, copolymers prepared from a monoethylenically unsaturated monocarboxylic acid and at least one comonomer, and mixtures of these photopolymers and copolymers.

The at least one comonomer may be selected from the group consisting of methacrylic acid, crotonic acid, maleic acid or maleic anhydride, itaconic acid, fumaric acid, citracronic acid and citracronic anhydride, vinylphosphonic acid, vinylsulfonic acid, 2-acrylamido-2-methylpropane-sulfonic acid (AMPS), (meth)acrylic acid derivatives, for example hydroxyethyl acrylate, hydroxypropyl acrylate, hydroxybutyl acrylate, (meth)acrylamide, vinylformamide, alkali metal (3-methacryloyloxy)propanesulfonate, dimethylaminoethyl acrylate, 2-acryloyloxyethyltrimethyl-ammonium chloride, dimethylamino methacrylate and polyethylene glycol methyl ether(meth)-acrylate. Particularly preferred the at least one comonomer is selected from the group consisting of maleic acid, maleic anhydride and 2-acrylamido-2-methyl-propanesulfonic acid (AMDS).

The monoethylenically unsaturated monocarboxylic acid and the at least one comonomer can be used in the form of free acids or else in completely or partly neutralized form for the preparation of the homopolymer and for the preparation of the copolymer.

The person skilled in the art knows that “free acids” usually means that the acidic groups of the monoethylenically unsaturated monocarboxylic acid and the at least one comonomer are present in their protonated form. For example carboxyl-groups are present as COOH. “Neutralized form” means that the acidic groups of the monoethylenically unsaturated monocarboxylic acid and the at least one comonomer are present in their deprotonated form, for example as a salt. Carboxyl-groups in their neutralized form for example means carboxylate groups (COO—). “Partly neutralized form” means that some of the acidic groups of the monoethylenically unsaturated monocarboxylic acid and the at least one comonomer are present as free acids and some are present in their neutralized form.

It should be clear that in case that the polyacrylic acid (PAA) is a copolymer, the monoethylenically unsaturated monocarboxylic acid differs from the at least one comonomer.

In case that the polyacrylic acid (PAA) is a copolymer, a copolymer selected from the group consisting of a poly(acrylic acid-maleic acid)-copolymer, a poly(acrylic acid-maleic anhydride)-copolymer or a poly(acrylic acid-2-acrylamido-2-methylpropanesulfonic acid)-copolymer is particularly preferred.

In a further preferred embodiment of the present invention the polyacrylic acid (PAA) is prepared from at least 50% by weight, preferably at least 80% by weight and more preferably at least 95% by weight of acrylic acid, based on the total amount of the acrylic acid and the at least one comonomer from which the polyacrylic acid (PAA) is prepared.

Methods for the preparation of polyacrylic acid (PAA) are known to the skilled person. Methods for its preparation are for example described in US 2012/0214041 A1 and in WO 2012/001092 A1. For example the polyacrylic acid (PAA) can be prepared by free-radical polymerization.

Suitable polymaleic acid comprises photopolymers prepared from a maleic acid, copolymers prepared from maleic acid and at least one comonomer, and mixtures of these photopolymers and copolymers. An expert is aware that maleic anhydride may be used to substitute maleic acid in part of in total.

The at least one comonomer in the polymaleic acid may be selected from the group consisting of crotonic acid, itaconic acid, fumaric acid, citracronic acid and citracronic anhydride, vinylphosphonic acid, vinylsulfonic acid, 2-acrylamido-2-methylpropanesulfonic acid (AMPS), (meth)acrylic acid derivatives, for example hydroxyethyl acrylate, hydroxypropyl acrylate, hydroxybutyl acrylate, (meth)acrylamide, vinylformamide, alkali metal (3-methacryloyloxy)-propanesulfonate, dimethylaminoethyl acrylate, 2-acryloyloxyethyltrimethylammonium chloride, dimethylamino methacrylate and polyethylene glycol methyl ether(meth)acrylate.

Examples for phosphonates are diethylenetriamine penta(methylene phosphonic acid) (DTPMP), amino tri(methylene phosphonic acid) (ATMP), 1-hydroxyethylidene-1,1- di-phosphonic acid (HEDP), 2-phosphonobutane-1,2,4-tricarboxylic acid (PBTC), ethylendiaminetetramethylene-phosphonic acid (EDTMP), hexamethylenediaminemethylen phosphonic acid (HMDTMP), hydroxyethylaminobismethylene phosphonic acid (HEMPA).

The salt water may comprise at least one salt selected from the group consisting of an alkaline metal salt, an alkaline earth metal salt and mixtures thereof. The salt water may comprise an additional salt, such as iron oxide.

For example, the salt water is process water, ground water, river water, brackish water, or sea water, wherein sea water is preferred. Suitable process water is cooling water in industrial plants or in power plants.

The salt water usually comprises the salt in a range from 0.001 to 10% by weight, preferably from 0.005 to 7.5% by weight, particularly preferably from 0.01 to 5% by weight, and in particular from 0.02 to 4% by weight.

Suitable alkaline metal salts are for example sodium sulfate (Na₂SO₄), sodium chloride (NaCl), sodium bromide (NaBr), sodium iodide (NaI), sodium carbonate (Na₂CO₃), potassium chloride (KCl), potassium bromide (KBr) and potassium iodide (KI).

Suitable alkaline earth metal salts are for example calcium fluoride (CaF₂), calcium sulfate (CaSO₄), calcium carbonate (CaCO3), magnesium fluoride (MgF₂), magnesium chloride (MgCl₂), magnesium bromide (MgBr₂), magnesium iodide (Mgl₂), magnesium sulfate (MgSO₄), magnesium carbonate (MgCO₃) and magnesium hydroxide (Mg(OH)₂).

The person skilled in the art knows that alkaline metal salts and alkaline earth metal salts generally dissociate in water. For example sodium chloride (NaCl) dissociates in water to give a sodium cation (Na⁺) and a chloride anion (Cl⁻), sodium carbonate (Na₂CO₃) dissociates in an aqueous medium to form two sodium cations (Na+) and a carbonate anion (CO₃²⁻) and calcium carbonate (CaCO₃) dissociates to give a calcium cation (Ca²⁺) and a carbonate anion (CO₃²⁻). A carbonate anion can also form bicarbonate (HCO₃⁻) in water. Therefore, alkaline metal salts and alkaline earth metal salts in water are usually present in their ionic form.

The salt water generally comprises at least 50% by weight, preferably at least 80% by weight and particularly preferably at least 90% by weight of water. In a preferred embodiment of the present invention the salt water comprises from 89.99% to 99.999% by weight of water, preferably from 92.494% to 99.995% by weight, particularly preferably from 94.996% to 99.99% by weight and more preferably from 95.998% to 99.98% by weight of water.

The salt water optionally comprises at least one further solvent. Generally, the salt water comprises at most 10% by weight, preferably at most 5% by weight, more preferably at most 2% by weight of the at least one further solvent. The at least one further solvent usually exhibits no miscibility gap with water. For example, the at least one solvent is a polar solvent, selected from the group consisting of methanol, ethanol, propanol and glycol.

The conductivity of the salt water is in one embodiment of the present invention in the range from 10 to 100000 μS/cm², preferably in the range from 10 to 30000 μS/cm² and particularly in the range from 10 to 500 μS/cm².

The temperature of the salt water is generally in the range from 0 to 100° C. Preferably, the temperature of the salt water is in the range from 5 to 95° C. and particularly preferably in the range from 10 to 50° C.

The salt water can have any pH-value. Preferably, the pH-value of the salt water is in the range from 5 to 9, particularly preferably in the range from 6 to 8 and more preferably in the range from 6.5 to 7.5.

The salt water may comprise the scale inhibitor in the range from 0.01 to 100 ppmw, preferably from 0.1 to 60 ppmw, particularly preferably from 0.1 to 40 ppmw and in particular from 0.1 to 20 ppmw. “ppmw” within the context of the present invention means parts per million by weight. 1 ppmw means 0.0001% by weight.

The method comprises the analysis with a calcium/magnesium ionselective electrode of
a) a dialyzed first sample of the salt water, and
b) a dialyzed second sample of the salt water which was supplemented with a known concentration of the scale inhibitor.

Optionally, the method may further comprise the analysis with a calcium/magnesium ionselective electrode of
c) a dialyzed third sample of an untreated salt water which is free of the scale inhibitor.

The dialyzed first sample of the salt water, the dialyzed second sample of the salt water, and optionally the dialyzed third sample of the untreated salt water, are together referred to as the dialyzed samples of the salt water.

The dialyzed samples of the salt water are usually obtainable by dialysis, such as by dialysis with a semi-permeable membrane.

The volume of the samples of the salt water which is subjected to dialysis is usually in the range from 1 to 2000 ml, preferably from 20 to 800 ml, and in particular from 50 to 400 ml.

In the dialysis at least a part of the salt is removed from the salt water by dialysis to give the dialyzed samples of the salt water. Typically at from 10 ppm to 5% of the salt is removed, preferably from 10 ppm to 1% and particularly preferably from 10 ppm to 100 ppm.

The dialysis may be achieved with a dialyzing unit, such as by pumping the salt water through the dialyzing unit. The principle of dialysis and suitable dialyzing units are known to the skilled person. Generally, a dialyzing unit comprises a buffer solution and at least one semi-permeable membrane. The semipermeable membrane separates the salt water from the buffer solution. The buffer solution has a lower concentration of the salt than the salt water. Therefore, to reach equilibrium between the concentration of the salt comprised in the salt water and the concentration of the salt comprised in the buffer solution, the salt comprised in the salt water diffuses through the semi-permeable membrane into the buffer solution.

To prevent the passage of the scale inhibitor from the salt water to the buffer solution through the semi-permeable membrane usually a semi-permeable membrane having a pore size smaller than the size of the scale inhibitor is used. The pore size of the semi-permeable membrane is for example ≤10000 Da, preferably 5000 Da and particularly preferably ≤1000 Da. In one embodiment the pore size of the semi-permeable membrane is in the range from 100 to 10000 Da, preferably from 200 to 5000 Da and particularly preferably from 300 to 1000 Da.

The semi-permeable membrane can have various forms, for example the form of a tube or of a cassette. The semi-permeable membrane can be made of any material that is suitable for the preparation of semi-permeable membranes and that allows the diffusion of the at least one electrolyte through the semipermeable membrane. Preferably, the semi-permeable membrane is made from cellulose nitrate, cellulose triacetate, cellulose acetate, regenerated cellulose, polyether sulfone, polyamide, polytetraflourethylene, polycarbonate or polyvinylchloride. Particularly preferred, the semi-permeable membrane is made from polyether sulfone.

Suitable buffer solutions are known to the skilled person. Preferably, the buffer solution comprises at least 90% by weight of demineralized water, based on the total amount of the buffer solution. In a particularly preferred embodiment, the buffer solution consists of deionized water. It should be clear to the person skilled in the art, that the composition of the buffer solution changes during the dialysis, as molecules of the salt diffuse into the buffer solution.

Usually, water such as deionized water is added to the dialyzed samples of the salt water. The deionized water is usually added in an amount so that the volume of the dialyzed sample of the salt water is the same as the volume of the sample of the salt water prior to dialysis. “The same” within the context of the present invention usually means a volume difference of ±10%, preferably of ±5% and particularly preferably of ±2%.

The dialyzed samples of the salt water usually have a conductivity of up to 200 μS/cm², preferably up to 100 μS/cm², and in particular up to 50 μS/cm². In another form the conductivity of the dialyzed samples of the salt water is in the range from 0.1 to 100 μS/cm², preferably in the range from 0.1 to 80 μS/cm², and in particular in the range from 0.1 to 30 μS/cm². It should be clear to the skilled person that the conductivity of the dialyzed samples of the salt water is lower than the conductivity of the salt water prior to dialysis.

The dialyzed samples of the salt water usually comprise the salt in a range from 0 to 100 ppmw, preferably from 0 to 70 ppmw, and in particular from 0 to 30 ppmw. It should be clear to the skilled person that the concentration of the salt comprised in the dialyzed samples of the salt water is lower than the concentration of the salt comprised in the salt water prior to dialysis.

The temperature of the dialyzed samples of the salt water is generally in the range from 0 to 100° C., preferably in the range from 5 to 95° C., and in particular in the range from 10 to 50° C.

The dialyzed samples of the salt water can have any pH-value. Typically, the pH-value of the dialyzed samples of the salt water is in the range from 5 to 9, preferably in the range from 6 to 8 and in particular in the range from 6.5 to 7.5.

The dialyzed first sample of the salt water typically comprises the scale inhibitor in a concentration which should be determined by the method according to the present invention.

Typically, the dialyzed first sample of the salt water comprises the scale inhibitor in the range from 0.01 to 100 ppmw, preferably from 0.1 to 60 ppmw, particularly preferably from 0.1 to 40 ppmw and in particular from 0.1 to 20 ppmw. “ppmw” within the context of the present invention means parts per million by weight. 1 ppmw means 0.0001% by weight.

The sample for the preparation of the dialyzed first sample of the salt water is usually collected after the treatment of the salt water with the scale inhibitor.

The dialyzed second sample of the salt water was supplemented with a known concentration of the scale inhibitor. Typically, the dialyzed second sample of the salt water is supplemented with known concentration of 0.1 to 50 ppm, preferably 0.5 to 10 ppm, and in particular 0.5 to 5 ppm of the scale inhibitor.

The scale inhibitor which is supplemented to the second sample should be the same as the scale inhibitor which concentration is determined by the method according to the invention.

The sample for the preparation of the dialyzed second sample of the salt water is usually collected after the treatment of the salt water with the scale inhibitor. Usually, it is collected at the same position as the dialyzed first sample.

The dialyzed third sample of an untreated salt water is free of the scale inhibitor. The untreated salt water is usually salt water, which was not treated with the scale inhibitor. The untreated salt water may comprise traces of the scale inhibitor which are already present in the untreated salt water before it entered the premises where the method according to the invention is made. In one form the untreated salt water may comprise up to 0.1 ppmw, preferably up to 0.01 ppmw of the scale inhibitor.

The sample for the preparation of the dialyzed third sample of the salt water is usually collected before the treatment of the salt water with the scale inhibitor.

The method comprises the analysis with a calcium/magnesium ionselective electrode.

The term “calcium/magnesium ionselective electrode” refers to a ionselective electrode, which is either selective to calcium, or to magnesium, or both to calcium and magnesium. In one form the ionselective electrode is selective to both calcium and magnesium. In another form the selectivity of the ionselective electrode to calcium, or to magnesium, or both to calcium and magnesium (preferably to both calcium and magnesium) is in the order of 3 to 4 decades higher compared to other divalent metal ions (e.g. Cu²⁺, Pb²⁺, Cd²⁺, Ba²⁺).

Ionselective electrodes in general, and calcium/magnesium ionselective electrode are known and commercially available, e.g. from OFS Online Fluid Sensoric GmbH, 07580 Ronneburg, Germany (www.water-monitoring.com).

Generally, an ionselective electrode includes a transducer which is able to convert the activity of a specific ion dissolved in a solution into a determinable signal, such as electrical potential, which can then be determined (e.g. via a voltmeter or pH meter).

The ionselective electrode may also include an ion-selective membrane, which preferentially allows one or more specific ions to pass through, relative to the other ions. Specific examples of ion-selective electrodes include, but are not limited to, electrodes containing glass membranes, crystalline membranes, or ion exchange resin membranes. In some cases, the performance of the ion-selective electrode may be enhanced by using a buffer, such as total ionic strength adjustment buffer which can be used to increase the ionic strength of a solution to a relatively high level.

The concentration of the scale inhibitor may be determined based on the analysis with the calcium/magnesium ionselective electrode of the dialyzed first sample of the salt water, and the dialyzed second sample of the salt water which was supplemented with a known concentration of the scale inhibitor. Optionally, the determination of the concentration may be additionally based on the analysis of the dialyzed third sample of an untreated salt water which is free of the scale inhibitor. Usually, this third data point helps to give more exact results.

The quantitative calculation of the concentration of the scale inhibitor is usually made by the standard addition method. The standard addition method is generally known, for example from DIN 32633 “Chemical analysis—Methods of Standard addition”.

The present invention also relates to a device for determining the concentration of the scale inhibitor in the salt water by the method according to the invention comprising the calcium/magnesium ionselective electrode, the dialyzing unit, and a dosage unit for supplementing the scale inhibitor to the second sample of the salt water.

Typically, the calcium/magnesium ionselective electrode, the dialyzing unit, and the dosage unit are connected via a circuit, e.g. plumbing.

The device may further comprise a conductivity sensor. The conductivity sensor may be connected to the dialyzing unit, e.g. via the circuit. The conductivity sensor may be used to determine the conductivity of conductivity of the salt water or of the dialyzed samples of the salt water.

Details of the calcium/magnesium ionselective electrode and the dialyzing unit are already given above.

The dosage unit for supplementing the scale inhibitor to the second sample of the salt water may comprise a pump which allows the controlled dosage of the scale inhibitor. The dosage unit may further comprise a reservoir of the scale inhibitor connected to the pump. The scale inhibitor in the reservoir should be the same as the scale inhibitor which concentration is determined by the method according to the invention.

The present invention also relates to a method for inhibiting incrustation in a plant which contains the salt water comprising the steps of
x) adding the scale inhibitor to the salt water at a desired concentration,
y) determining the actual concentration of the scale inhibitor in the salt water by the method according to the invention, and
z) adding further scale inhibitor to the salt water to adjust the desired concentration.

Typical plants which contain the salt water, where the incrustation is inhibited, are desalination plants for sea water (e.g. thermic desalination plants or reversed osmosis desalination plants), cooling towers in industrial plants, cooling circuits in industrial plants, or boiler water treatment in industrial plants, waste water treatment plants, heat exchanger used in water cycles, evaporators used in zero liquid discharge system, evaporators used in sugar mills or paper mills.

In step x) the scale inhibitor to the salt water at a desired concentration. The desired concentration of the scale inhibitor in the salt water is usually in the range from 0.01 to 100 ppmw, preferably from 0.1 to 60 ppmw, particularly preferably from 0.1 to 40 ppmw and in particular from 0.1 to 20 ppmw.

Over time the desired concentration of the scale inhibitor in the salt water may decrease. The actual concentration may be lower than the desired concentration of the scale inhibitor. In step y) the actual concentration of the scale inhibitor in the salt water is determined with the method according to the invention.

In step z) further scale inhibitor is added to the salt water to adjust the desired concentration. The amount of the further scale inhibitor usually depends on the results of the concentration as determined in step y).

The present invention offer various advantages: It allows the exact determination of the concentration of the scale inhibitor, it is very reliable, and cheap. The concentration of the scale inhibitor can be determined in bypass procedure.

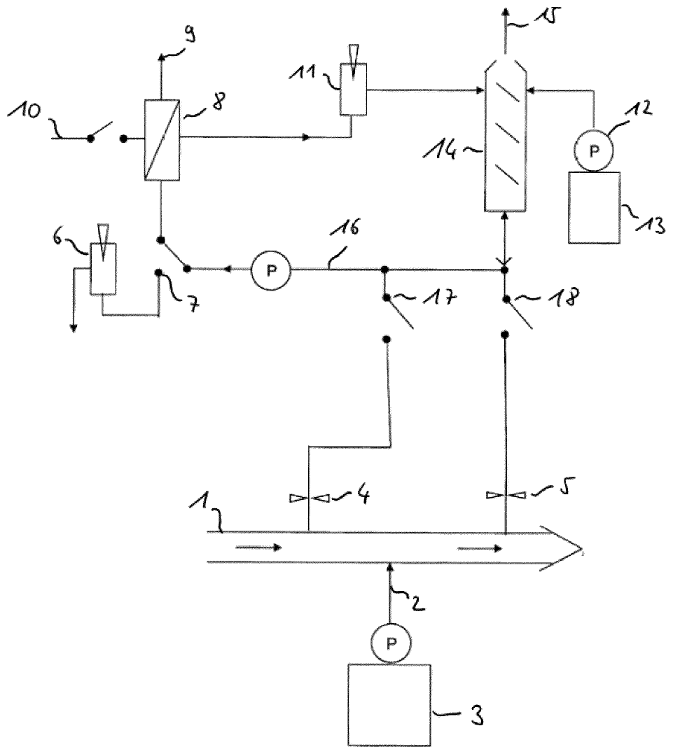

FIG. 1 show a typical example of a method and device according to the invention:

The salt water flows through a plant in a pipeline 1. The flow direction of the salt water is indicated with arrows.

The scale inhibitor is added to the salt water at the inlet 2 into the pipeline 1, usually with a pump P from a storage tank 3 containing the scale inhibitor.

At outlet 4 a sample can be taken to prepare the dialyzed third sample of the untreated salt water. The outlet 4 is usually before the inlet 2, where the scale inhibitor is added.

At outlet 5 a sample can be taken to prepare the dialyzed first and second sample of the salt water. The outlet 5 is usually after the inlet 2, where the scale inhibitor is added.

The calcium/magnesium ionselective electrode 6 is connected by a valve 7 to a circuit 16 where the samples from outlet 4 and outlet 5 are added at the valves 17 and 18, respectively.

The dialyzing unit 8 has an outlet 9 and an inlet 10 for the deionized water.

The conductivity sensor 11 is connected via the circuit 16 to the dialyzing unit 8.

The dialyzed second sample of the salt water can be supplemented with a known concentration of the scale inhibitor by a dosage unit 12 which comprises a pump P and a reservoir 13 of the scale inhibitor. The dosage unit 12 is connected to a mixer 14 to ensure a good mixing. The mixer may contain an outlet 15 to empty the whole circuit 16.

1 . A method for determining a concentration of a scale inhibitor in a salt water comprising an analysis with a calcium/magnesium ionselective electrode of
a) a dialyzed first sample of the salt water, and
b) a dialyzed second sample of the salt water which was supplemented with a known concentration of the scale inhibitor.

2. The method according to claim 1 further comprising the analysis with a calcium/magnesium ionselective electrode of
c) a dialyzed third sample of an untreated salt water which is free of the scale inhibitor.

3. The method according to claim 1 wherein the salt water comprises at least one salt selected from the group of an alkaline metal salt, an alkaline earth metal salt and mixtures thereof.

4. The method according to claim 1 wherein the salt water comprises from 0.001 to 10% by weight the salt, based on the total weight of the salt water.

5. The method according to claim 1 wherein the salt water is chosen from process water, ground water, river water, brackish water, or sea water.

6. The method according to claim 1 wherein the dialyzed samples of the salt water are obtainable by dialysis with a semi-permeable membrane which has a pore size of up to 10000 Da.

7. The method according to claim 1 wherein the conductivity of the dialyzed samples of the salt water is up to 200 μS/cm².

8. The method according to claim 1 wherein the dialyzed second sample of the salt water is supplemented with known concentration of 0.1 to 50 ppm of the scale inhibitor.

9. The method according to claim 1 wherein the scale inhibitor has a number average molecular weight Mₙ in the range from 200 to 250000 g/mol.

10. The method according to claim 1 wherein the scale inhibitor is a polycarboxylic acid or a phosphonate.

11. The method according to claim 1 wherein the scale inhibitor is a polyacrylic acid selected from photopolymers prepared from a monoethylenically unsaturated monocarboxylic acid, copolymers prepared from a monoethylenically unsaturated monocarboxylic acid and at least one comonomer, and mixtures of these photopolymers and copolymers.

12. The method according to claim 11 wherein the scale inhibitor comprises a copolymer prepared from a monoethylenically unsaturated monocarboxylic acid and at least one comonomer, and wherein the at least one comonomer is selected from the group consisting of methacrylic acid, crotonic acid, maleic acid or maleic anhydride, itaconic acid, fumaric acid, citracronic acid and citracronic anhydride, vinylphosphonic acid, vinylsulfonic acid, 2-acrylamido-2-methylpropanesulfonic acid (AMPS), (meth)acrylic acid derivatives, (meth)acrylamide, vinylformamide, alkali metal (3-methacryloyloxy)propane-sulfonate, dimethylaminoethyl acrylate, 2-acryloyloxyethyltrimethylammonium chloride, dimethylamino methacrylate, polyethylene glycol methyl ether(meth)acrylate, or a combination thereof.

13. A method for inhibiting incrustation in a plant which contains a salt water comprising the steps of
x) adding a scale inhibitor to the salt water at a desired concentration,
y) determining the actual concentration of the scale inhibitor in the salt water as defined claim 1, and
z) adding further scale inhibitor to the salt water to adjust the desired concentration.

14. The method according to claim 13 wherein the plant is chosen from a desalination plant for sea water, a cooling tower in an industrial plant, a cooling circuit in an industrial plant, or a boiler water treatment in an industrial plant.

15. A device for determining a concentration of a scale inhibitor in a salt water by the method as defined in claim 1, wherein the device comprises a calcium/magnesium ion selective electrode (6), a dialyzing unit (8), and a dosage unit (12) for supplementing the scale inhibitor to the second sample of the salt water.

16. The method according to claim 1 wherein the conductivity of the dialyzed samples of the salt water is up to 100 μS/cm².

17. The method according to claim 1 wherein the conductivity of the dialyzed samples of the salt water is up to 50 μS/cm².

18. The method according to claim 1 wherein the dialyzed second sample of the salt water is supplemented with known concentration of 0.5 to 10 ppm of the scale inhibitor.

19. The method according to claim 1 wherein the dialyzed second sample of the salt water is supplemented with known concentration of 1 to 3 ppm of the scale inhibitor.

20. The method according to claim 11 wherein the scale inhibitor comprises a copolymer prepared from a monoethylenically unsaturated monocarboxylic acid and at least one comonomer, and wherein the at least one comonomer is selected from the group of maleic acid, maleic anhydride or 2-acrylamido-2-methyl-propanesulfonic acid.
